(19) United States
(12) Patent Application Publication
(10) Pub. No.: US 2024/0082550 A1
(43) Pub. Date: Mar. 14, 2024

(54) GUIDE WIRE

(71) Applicant: ASAHI INTECC CO., LTD., Seto-shi (JP)

(72) Inventors: Nami MAWATARI, Seto-shi (JP); Rikuto Omi, Seto-shi (JP)

(73) Assignee: ASAHI INTECC CO., LTD., Seto-shi (JP)

(21) Appl. No.: 18/518,758

(22) Filed: Nov. 24, 2023

Related U.S. Application Data
(63) Continuation of application No. PCT/JP22/19715, filed on May 9, 2022.

(30) Foreign Application Priority Data
May 25, 2021 (JP) 2021-087943

(51) Int. Cl. A61M 25/09

(52) U.S. Cl. CPC A61M 25/09; A61M 2025/09083; A61M 2025/09175

(57) ABSTRACT

A guide wire including a core shaft that has a distal end portion and a proximal end portion. The distal end portion includes a first region having a flat shape in cross section, and a second region having a circular shape in cross section and having flexural rigidity higher than that of the first region. The first region and the second region are provided in the distal end portion in the stated order from a most distal end side, and at least two or more sets of the first region and the second region are provided at the distal end portion. Flat directions of the respective first regions are all identical.

### CROSS REFERENCE TO RELATED APPLICATIONS

This application is a continuation application of International Application No. PCT/JP2022/019715, filed May 9, 2022, which claims priority to Japanese Patent Application No. 2021-087943, filed May 25, 2021. The contents of these applications are incorporated herein by reference in their entirety.

### TECHNICAL FIELD

This application relates to a guide wire.

### BACKGROUND

When treating a stenosis occurring in a blood vessel such as a coronary artery surrounding the heart, or when treating a completely blocked site in a blood vessel (e.g., chronic total occlusion: CTO, etc.) due to progressive calcification, a guide wire for guiding a therapeutic implement such as a balloon catheter is inserted into a blood vessel prior to the therapeutic implement.

As the guide wire, for example, a guide wire has been proposed in which a distal end portion of a core wire has a tapered portion and a flat portion continuously extending from the tapered portion toward a distal end side, thereby improving flexibility of the distal end portion (for example, see FIG. 2 (b) of JP 2004-154286 A).

### SUMMARY

However, in the guide wire of Patent Literature 1, since the distal end portion has a flat shape, the shaping performance of reforming (shaping) the distal end portion at a medical site and a direction in which the distal end portion is bent when the distal end portion is pushed into a lesion can be matched, but transmission performance of rotation torque is reduced.

As one of methods for securing the torque transmission performance, it is conceivable to form the distal end portion into a round bar shape (see FIG. 2 (a) of JP 2004-154286 A). However, when the distal end portion is formed into the round bar shape, not only the reforming at the time of shaping becomes three dimensional, but also the distal end portion may be bent in a direction different from an intended direction (so-called three-dimensional deformation) when the distal end is pushed into a lesion.

One object of the present disclosure is to provide a guide wire having good torque transmission performance while ensuring shaping performance and bendability of a distal end portion.

A guide wire according to an aspect of the disclosure includes a core shaft that has a distal end portion and a proximal end portion. The distal end portion includes: a first region having a flat shape in cross section; and a second region having a circular shape in cross section and having flexural rigidity higher than that of the first region. The first region and the second region are provided in the distal end portion in the stated order from a most distal end side, and at least two or more sets of the first region and the second region are provided at the distal end portion. Flat directions of the respective first regions are all identical.

### BRIEF DESCRIPTION OF DRAWINGS

FIG. 1 is a schematic view of a guide wire according to a first embodiment.

FIG. 2 is a longitudinal sectional view of a first region of a core shaft.

FIG. 3 is a schematic view of a guide wire according to a second embodiment.

FIG. 4 is a schematic view of a guide wire according to a modification of the second embodiment.

FIG. 5 is a schematic view of a jig used for a rotational performance evaluation.

FIG. 6 is a graph illustrating a relationship between a hand angle (input angle) and a distal end angle (output angle) in the rotational performance evaluation of an example and a comparative example.

### DETAILED DESCRIPTION

Hereinafter, a guide wire according to an embodiment of the present disclosure will be described with reference to the drawings, but the disclosed embodiments are not limited only to the embodiments illustrated in the drawings. In the present disclosure, a distal end refers to an end portion of the guide wire where a distal tip is located, and a proximal end refers to an end portion on a side opposite to the distal end.

### First Embodiment

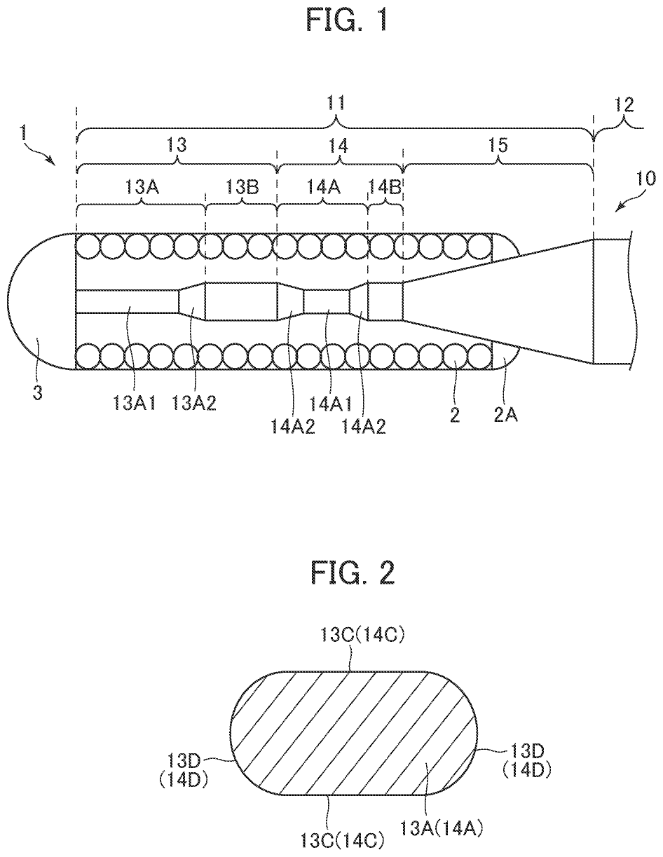

FIG. 1 is a schematic view of a guide wire 1 according to the first embodiment. As illustrated in FIG. 1, the guide wire 1 includes a core shaft 10, a coil body 2, and a distal tip 3.

The core shaft 10 is a shaft extending from the proximal end to the distal end of the guide wire 1. The core shaft 10 has a distal end portion 11 located on a distal end side (left side in FIG. 1) and a proximal end portion 12 located on a proximal end side (right side in FIG. 1) with respect to the distal end portion 11. The distal end portion 11 has a first distal end portion 13, a second distal end portion 14, and a tapered portion 15 in this order from the distal end side.

The first distal end portion 13 has a first region 13A having a flat shape in cross section and a second region 13B having a circular shape in cross section and having flexural rigidity higher than that of the first region 13A. The first region 13A has a flat plate region 13A1 and a tapered region 13A2. The flat plate region 13A1 is formed into a flat plate shape by, for example, pressing a cylindrical portion having the same radius as an outer diameter of the second region 13B. In this example, a press direction of the flat plate region 13A1 is an up-down direction in FIG. 1, and a long axis (plate width direction) of the flat plate region 13A1 is a direction perpendicular to a paper surface of FIG. 1.

The tapered region 13A2 connects the second region 13B and the flat plate region 13A1, and the outer diameter of the tapered region 13A2 gradually decreases from the second region 13B toward the flat plate region 13A1. FIG. 2 is a longitudinal sectional view of the first region 13A. As illustrated in FIG. 2, in this embodiment, since a round rod body is pressed from the up-down direction, press surfaces (side surfaces in a plate thickness direction in FIG. 1) of the flat plate region 13A1 and the tapered region 13A2 are smooth surfaces 13C. In addition, since side surfaces in the plate width direction are unpressed portions, the side surfaces are circular arc surfaces 13D.

The second distal end portion 14 has a first region 14A having a flat shape in cross section and a second region 14B having a circular shape in cross section and having flexural rigidity higher than that of the first region 14A. The first region 14A has a flat plate region 14A1 and two tapered regions 14A2. The flat plate region 14A1 is formed into a flat plate shape by, for example, pressing a cylindrical portion having the same radius as the outer diameter of the second region 14B.

One tapered region 14A2 connects an end portion (left end in FIG. 1) of the flat plate region 14A1 and an end portion (right end in FIG. 1) of the second region 13B, and in this example, the outer diameter of the one tapered region 14A2 gradually decreases from the second region 13B toward the flat plate region 14A1. The other tapered region 14A2 connects an end portion (right end in FIG. 1) of the flat plate region 14A1 and an end portion (left end in FIG. 1) of the second region 14B, and in this embodiment, the outer diameter of the other tapered region 14A2 gradually decreases from the second region 14B toward the flat plate region 14A1. As described above, the distal end portion 11 is provided with at least two or more sets of the first regions 13A and 14A and the second regions 13B and 14B.

In this embodiment, similarly to the flat plate region 13A1 and the tapered region 13A2, since a round rod body is pressed from the up-down direction, press surfaces (side surfaces in the plate thickness direction in FIG. 2) of the flat plate region 14A1 and the tapered region 14A2 are smooth surfaces 14C. In addition, since side surfaces in the plate width direction are unpressed portions, the side surfaces are circular arc surfaces 14D.

Flat directions of the first region 13A (flat plate region 13A1) and the first region 14A (flat plate region 14A1) are all configured to be identical. That is, the first region 13A and the first region 14A are configured such that longitudinal directions of the cross sections thereof are parallel to each other. Therefore, in the first region 13A and the first region 14A, bending directions thereof are identical. The first region 13A is slightly shaped in the bending direction thereof before use.

The flat shapes of the first regions 13A and 14A are shapes including an oval shape, an elliptical shape, and the like in which at least the press surface is a smooth surface, and are shapes in which the bending direction is determined to be a specific direction when the guide wire 1 is used.

The flexural rigidity of the flat plate regions 13A1 and 14A1 is configured to be equal, and the flexural rigidity of the second regions 13B and 14B is configured to be equal. That is, the thicknesses of the flat plate regions 13A1 and 14A1 are configured to be equal, and the outer diameters of the second regions 13B and 14B are configured to be equal.

Examples of a material constituting the core shaft 10 include a stainless steel such as SUS304, and a metallic material such as a Ni—Ti alloy and a Co—Cr alloy. A total length of the core shaft 10 is, for example, 1,800 to 3,000 mm, a length of the first region 13A is, for example, 5 to 20 mm, a length of the second region 13B is, for example, 3 to 7 mm, a length of the first region 14A is, for example, 3 to 7 mm, a length of the second region 14B is, for example, 5 to 10 mm, and a length of the tapered portion 15 is, for example, 30 to 100 mm. The thicknesses of the flat plate regions 13A1 and 14A1 are, for example, 0.04 to 0.07 mm, and the outer diameters of the second regions 13B and 14B are, for example, 0.06 to 0.10 mm.

The coil body 2 is provided around the distal end portion 11 of the core shaft 10. The coil body 2 is formed in a hollow cylindrical shape by spirally winding a metal wire around the core shaft 10. A distal end of the coil body 2 is joined to the distal tip 3, and a proximal end of the coil body 2 is joined to the tapered portion 15 by a joint portion 2A. The joint portion 2A is made of, for example, a brazing material (aluminum alloy braze, silver braze, gold braze, etc.), a metal solder (Ag—Sn alloy, Au—Sn alloy, etc.), and an adhesive (epoxy-based adhesive, etc.), or the like.

The metal wire constituting the coil body 2 is one or a plurality of solid wires or one or a plurality of strands. A diameter of the metal wire is, for example, 0.01 to 0.10 mm. Examples of a material constituting the metal wire of the coil body 2 include a stainless steel such as SUS316, a super-elastic alloy such as a Ni—Ti alloy, and radio-opaque metal such as platinum and tungsten.

The distal tip 3 has a substantially hemispherical shape, is provided at the distal end of the guide wire 1, and joins the distal end of the core shaft 10 and the distal end of the coil body 2. The distal tip 3 is made of, for example, a brazing material (aluminum alloy braze, silver braze, gold braze, etc.), a metal solder (Ag—Sn alloy, Au—Sn alloy, etc.), and an adhesive (epoxy-based adhesive, etc.), or the like.

As described above, according to the guide wire 1 of the first embodiment, the distal end portion 11 of the core shaft 10 includes the first regions 13A and 14A each having a flat shape in cross section and the second regions 13B and 14B each having a circular shape in cross section and each having a flexural rigidity higher than that of the first regions 13A and 14A, and the distal end portion 11 is provided with the first region 13A (14A) and the second region 13B (14B) in the stated order from a most distal end side, and is provided with at least two or more sets of the first region and the second region (e.g., 13A and 13B, and 14A and 14B), and the flat directions of the respective first regions 13A and 14A are all identical.

According to the above configuration, since the flat directions of the respective first regions 13A and 14A are all identical, the bending directions of the first regions 13A and 14A can be set to the same direction, and the three-dimensional deformation of the distal end portion 11 can be reliably suppressed. Further, since the distal end portion 11 has the second regions 13B and 14B each having a circular shape in cross section, it is possible to ensure torque transmission performance. In this way, it is possible to provide the guide wire 1 capable of securing the shaping performance and the torque transmission performance.

### Second Embodiment

Next, a guide wire according to a second embodiment of the present disclosure will be described with reference to the drawings. The same members as those of the guide wire 1 of the first embodiment are denoted by the same reference numerals, description thereof will be omitted, and configurations different from that of the guide wire 1 of the first embodiment will be described.

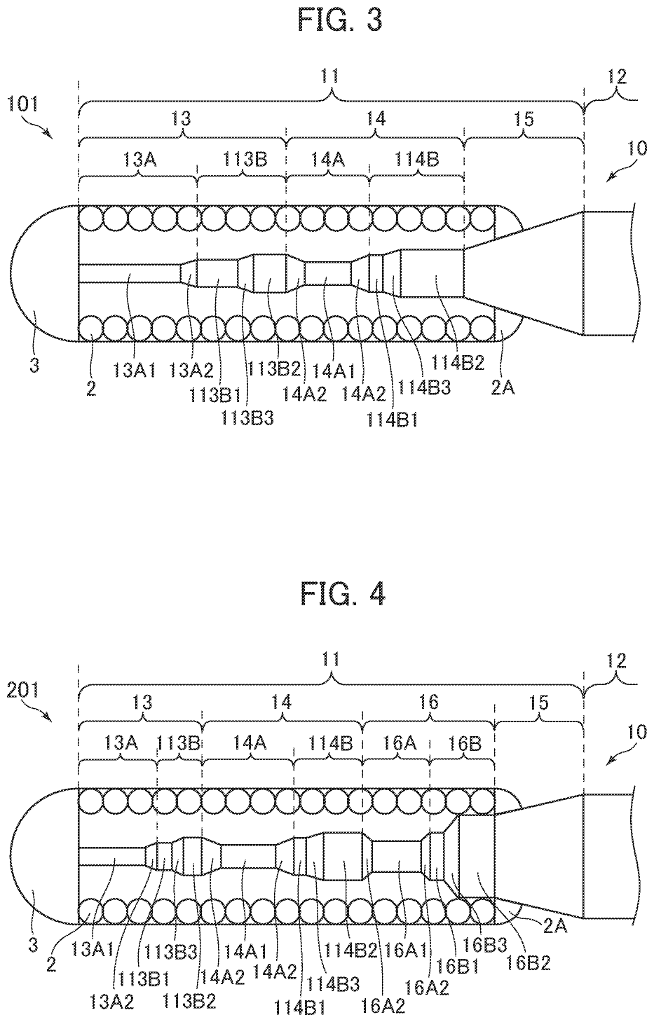

FIG. 3 is a schematic view of a guide wire 101 according to the second embodiment. As illustrated in FIG. 3, the configurations of the second regions 113B and 114B are different from the configurations of the second regions 13B and 14B of the first embodiment.

The second region 113B has a first portion 113B1, a second portion 113B2, and a tapered portion 113B3. The first portion 113B1 has a circular shape in cross section, and a distal end (left end in FIG. 3) of the first portion 113B1 is connected to a proximal end (right end in FIG. 3) of the tapered region 13A2 of the first region 13A. The second portion 113B2 has a circular shape in cross section, and a proximal end (right end in FIG. 3) of the second portion 113B2 is connected to a distal end (left end in FIG. 3) of the tapered region 14A2 on a distal end side of the first region 14A. The tapered portion 113B3 is located between the first portion 113B1 and the second portion 113B2, and connects a proximal end (right end in FIG. 3) of the first portion 113B1 and a distal end (left end in FIG. 3) of the second portion 113B2. In this example, an outer diameter of the tapered portion 113B3 gradually decreases from the second portion 113B2 toward the first portion 113B1.

The second portion 113B2 has an outer diameter larger than that of the first portion 113B1. That is, the second portion 113B2 is configured to have flexural rigidity higher than that of the first portion 113B1, and a rigid gap is formed by the two portions.

The second region 114B has a first portion 114B1, a second portion 114B2, and a tapered portion 114B3. The first portion 114B1 has a circular shape in cross section, and a distal end (left end in FIG. 3) of the first portion 114B1 is connected to a proximal end (right end in FIG. 3) of the tapered region 14A2 on a proximal end side (right side in FIG. 3) of the first region 14A. An outer diameter of the first portion 114B1 is the same as an outer diameter of the second portion 113B2 of the second region 113B. The second portion 114B2 has a circular shape in cross section, and a proximal end (right end in FIG. 3) of the second portion 114B2 is connected to a distal end (left end in FIG. 3) of the tapered portion 15. The tapered portion 114B3 is located between the first portion 114B1 and the second portion 114B2, and connects a proximal end (right end in FIG. 3) of the first portion 114B1 and a distal end (left end in FIG. 3) of the second portion 114B2. In this example, an outer diameter of the tapered portion 114B3 gradually decreases from the second portion 114B2 toward the first portion 114B1.

The second portion 114B2 has an outer diameter larger than that of the first portion 114B1. That is, the second portion 114B2 is configured to have flexural rigidity higher than that of the first portion 114B1, and a rigid gap is formed by the two portions. In the present embodiment, the flat plate region 14A1 of the first region 14A is configured to be thicker than the flat plate region 13A1 of the first region 13A.

The length of the first region 13A is, for example, 5 to 10 mm, the length of the first portion 113B1 is, for example, 0 to 2 mm, the length of the second portion 113B2 is, for example, 5 to 7 mm, the length of the tapered portion 113B3 is, for example, 1 to 2 mm, the length of the first region 14A is, for example, 5 to 10 mm, the length of the first portion 114B1 is, for example, 0 to 2 mm, the length of the second portion 114B2 is, for example, 5 to 7 mm, and the length of the tapered portion 114B3 is, for example, 1 to 2 mm. The thickness of the flat plate region 13A1 is, for example, 0.04 to 0.07 mm, and the thickness of the flat plate region 14A1 is, for example, 0.11 to 0.14 mm. The outer diameter of the first portion 113B1 is, for example, 0.06 to 0.10 mm, the outer diameter of the second portion 113B2 is, for example, 0.15 to 0.20 mm, the outer diameter of the first portion 114B1 is, for example, 0.15 to 0.20 mm, and the outer diameter of the second portion 114B2 is, for example, 0.25 to 0.34 mm.

According to the guide wire 101 of the present embodiment, substantially the same effects as those of the guide wire 1 of the first embodiment can be obtained. Furthermore, in the guide wire 101 of the present embodiment, the second regions 113B and 114B respectively have the first portions 113B1 and 114B1, the second portions 113B2 and 114B2 that are located on the proximal end side of the first portions 113B1 and 114B1 and have outer diameters larger than those of the first portions 113B1 and 114B1, and the tapered portions 113B3 and 114B3 that are located between the first portions 113B1 and 114B1 and the second portions 113B2, 114B2 and are decreasingly tapered from the second portions 113B2 and 114B2 toward the first portions 113B1 and 114B 1.

Therefore, in the distal end portion 11, a first rigid gap is formed between the first portion 113B1 and the second portion 113B2 on the distal end side, and a second rigid gap is formed between the first portion 114B1 and the second portion 114B2 on the proximal end side. In this embodiment, it is set so that the first rigid gap is smaller than the second rigid gap.

Thus, when a lesion consists of relatively soft tissue, the first rigid gap suppresses advancement of a knuckle in the first region 13A. When the lesion consists of a relatively hard tissue, even when the distal end of the guide wire 101 comes into contact with the lesion and the advancement of the knuckle in the first region 13A does not stop, the advancement of the knuckle can be stopped by the second rigid gap. At that time, the respective flat plate regions 13A1 and 14A1 are all flattened in the same direction, and the bending directions thereof are aligned in the same direction, so that the three-dimensional deformation of the distal end portion 11 can be reliably suppressed. In this way, it is possible to provide the guide wire 101 which can also deal with a hard lesion. In the second regions 113B and 114B, the rigidity is increased toward the proximal end side, so that the hardness of the lesion can be known by the portion at which the knuckle stops.

Next, a guide wire according to a modification of the second embodiment will be described with reference to the drawings. FIG. 4 is a schematic view of a guide wire 201 according to a modification of the second embodiment. As illustrated in FIG. 4, the guide wire 201 further has a third distal end portion 16 at the distal end portion 11 of the core shaft 10 in addition to the first distal end portion 13 and the second distal end portion 14. The third distal end portion 16 is provided between the second distal end portion 14 and the tapered portion 15. In the present modification, lengths of the first distal end portion 13 and the second distal end portion 14 in the axial direction of the core shaft 10 are configured to be shorter than the lengths of the first distal end portion 13 and the second distal end portion 14 in the axial direction of the core shaft 10 in the second embodiment of FIG. 3.

The third distal end portion 16 has a first region 16A having a flat shape in cross section and a second region 16B having a circular shape in cross section and having flexural rigidity higher than that of the first region 16A.

The first region 16A has a flat plate region 16A1 and two tapered regions 16A2. The flat plate region 16A1 is formed into a flat plate shape by, for example, pressing a cylindrical portion having the same radius as the outer diameter of the second region 16B.

One tapered region 16A2 connects an end portion of the flat plate region 16A1 (left end in FIG. 4) and an end portion of the second portion 114B2 (right end in FIG. 4), and in this example, the outer diameter of the one tapered regions 16A2 gradually decreases from the second portion 114B2 toward the flat plate region 16A1. The other tapered region 16A2 connects an end portion (right end in FIG. 4) of the flat plate region 16A1 and an end portion (left end in FIG. 4) of the second region 16B, and in this example, the outer diameter of the other tapered region 16A2 gradually decreases from the second region 16B toward the flat plate region 16A1. As described above, three sets of the first regions 13A, 14A, and 16A and the second regions 113B, 114B, and 16B are provided in the distal end portion 11.

In this embodiment, similarly to the flat plate region 13A1 and the tapered region 13A2, since a round rod body is pressed from the up-down direction, press surfaces (side surfaces in the plate thickness direction in FIG. 4) of the flat plate region 16A1 and the tapered region 16A2 are smooth surfaces. In addition, since side surfaces in the plate width direction are unpressed portions, the side surfaces are circular arc surfaces.

The flat directions of the first region 13A (flat plate region 13A1), the first region 14A (flat plate region 14A1), and the first region 16A (flat plate region 16A1) are all configured to be identical. That is, the first region 13A, the first region 14A, and the first region 16A are configured such that longitudinal directions of the cross sections thereof are parallel to one another. Therefore, in the first region 13A, the first region 14A, and the first region 16A, bending directions thereof are identical.

The second region 16B has a first portion 16B1, a second portion 16B2, and a tapered portion 16B3. The first portion 16B1 has a circular shape in cross section, and a distal end (left end in FIG. 4) of the first portion 16B1 is connected to a proximal end (right end in FIG. 4) of the tapered region 16A2 on the proximal end side (right side in FIG. 4) of the first region 16A. An outer diameter of the first portion 16B1 is the same as the outer diameter of the second portion 114B2 of the second region 114B. The second portion 16B2 has a circular shape in cross section, and a proximal end (right end in FIG. 4) of the second portion 16B2 is connected to a distal end (left end in FIG. 4) of the tapered portion 15. The tapered portion 16B3 is located between the first portion 16B1 and the second portion 16B2, and connects a proximal end (right end in FIG. 4) of the first portion 16B1 and a distal end (left end in FIG. 4) of the second portion 16B2. In this example, the tapered portion 16B3 is configured to be tapered such that the outer diameter thereof gradually decreases from the second portion 16B2 toward the first portion 16B1.

The second portion 16B2 has an outer diameter larger than that of the first portion 16B1. That is, the second portion 16B2 is configured to have flexural rigidity higher than that of the first portion 16B1, and a rigid gap is formed by the two portions. In the present modification, the flat plate region 16A1 of the first region 16A is configured to be thicker than the flat plate region 14A1 of the first region 14A.

According to the guide wire 201 of the present modification, substantially the same effects as those of the guide wire 101 of the second embodiment can be obtained. Additionally, the guide wire 201 of the present embodiment has a second region 16B in addition to each of the second regions 113B and 114B. The second region 16B has a first portion 16B1, a second portion 16B2 that is located on the proximal end side of the first portion 16B1 and has an outer diameter larger than that of the first portion 16B1, and a tapered portion 16B3 that is located between the first portion 16B1 and the second portion 16B2 and is decreasingly tapered from the second portion 16B2 toward the first portion 16B1.

Therefore, in the distal end portion 11, in addition to the first rigid gap and the second rigid gap, a third rigid gap is formed between the first portion 16B1 and the second portion 16B2 on the most proximal end side. In this embodiment, it is set so that the first rigidity gap is smaller than the second rigidity gap, and the second rigidity gap is smaller than the third rigidity gap.

Thus, when the lesion consists of a hard tissue, in the guide wire 201, even when the second distal end portion 14 thereof comes into contact with the lesion and the advancement of the knuckle in the first region 14A does not stop, the advancement of the knuckle can be stopped by the third rigid gap. At that time, the respective flat plate regions 13A1, 14A1, and 16A1 are all flattened in the same direction, and the bending directions thereof are aligned in the same direction, so that the three-dimensional deformation of the distal end portion 11 can be reliably suppressed. In this way, it is possible to provide the guide wire 201 which can also deal with a hard lesion. In the second regions 113B, 114B, and 16B, the rigidity is increased toward the proximal end side, so that the hardness of the lesion can be known by the portion at which the knuckle stops.

Next, results of rotational performance evaluation of the guide wire 101 of the present embodiment will be described. The dimensions of the guide wire 101 used as an example are as follows: the length of the first region 13A is 10 mm, the length of the second region 113B is 6 mm, the length of the first region 14A is 5 mm, the length of the second region 114B is 6 mm, the thickness of the flat plate region 13A1 is 0.04 mm, the outer diameter of the first portion 113B1 is 0.06 mm, the outer diameter of the second portion 113B2 is 0.10 mm, the thickness of the flat plate region 14A1 is 0.055 mm, the outer diameter of the first portion 114B1 is 0.10 mm, the outer diameter of the second portion 114B2 is 0.17 mm, and the outer diameter of the proximal end portion 12 is 0.33 mm. The guide wire used as the comparative example had the same dimensions on the proximal end side with respect to the tapered portion 114B3 as those of the guide wire 101 of the example, and an entire portion from the first portion 114B1 to the flat plate region 13A1 is formed in a flat plate shape having a thickness of 0.05 mm.

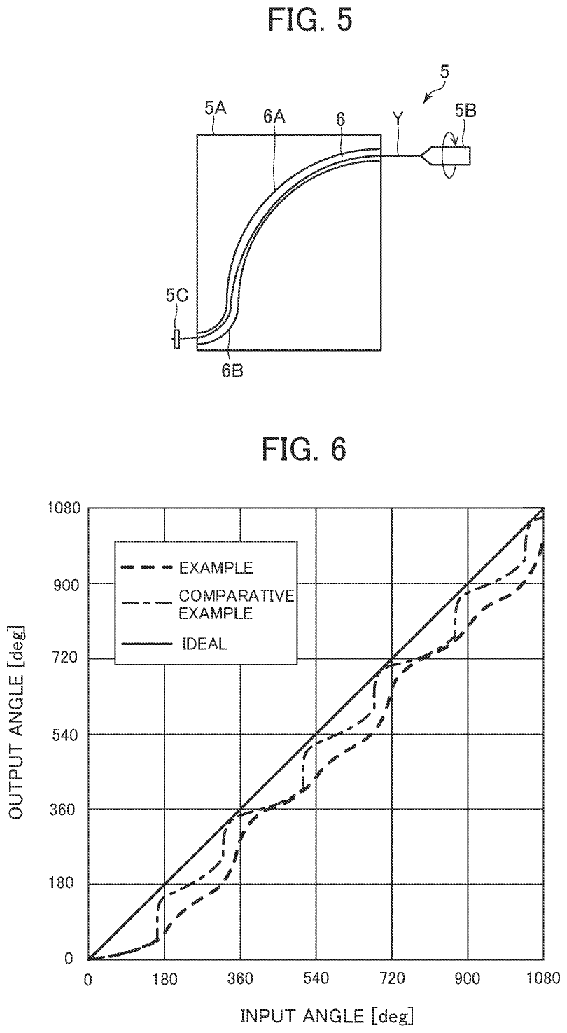

FIG. 5 is a schematic view of a jig 5 used for evaluating the rotational performance. The jig 5 includes a wire insertion portion 5A, an input portion 5B, and an output portion 5C. The wire insertion portion 5A is made of a resin (for example, a transparent acrylic plate) and has a groove 6 formed therein. The groove 6 is opened at a side surface of the wire insertion portion 5A. A width of the groove 6 is 1.0 mm. The groove 6 has a plurality of circular arc portions 6A and 6B. A curvature radius of the circular arc portion 6A is 70 mm, and a curvature radius of the circular arc portion 6B is 5 mm. The input portion 5B has a substantially cylindrical shape, is provided in a rotatable manner, and is connected to a proximal end of a guide wire Y. A distal end of the guide wire Y is connected to the output portion 5C.

The jig 5 was used to measure followability (following angle) of the output portion 5C when the input portion 5B was rotated counterclockwise. The results are illustrated in FIG. 6. FIG. 6 is a graph illustrating a relationship between a hand angle (input angle) and a distal end angle (output angle) in a rotational performance evaluation of the example and the comparative example.

As illustrated in FIG. 6, the measurement result of the guide wire 101 of the example is a shape close to a linear shape, resulting in a good rotation followability. On the other hand, the measurement result of the guide wire of the comparative example has a wave shape as a whole, resulting in a poor rotation followability. As described above, the guide wire 101 of the present embodiment has an excellent rotation followability.

It should be noted that the present disclosure is not limited to the configurations of the aforementioned embodiments but defined by the scope of claims. The present disclosure is intended to include all modifications within the meaning and scope equivalent to those of claims.

For example, in the first embodiment, the thicknesses of the flat plate regions 13A1 and 14A1 are configured to be identical, but the flat plate region 14A1 may be configured to be thicker than the flat plate region 13A1. Although the outer diameters of the second regions 13B and 14B are configured to be identical, the outer diameter of the second region 14B may be configured to be larger than the outer diameter of the second region 13B. In the above embodiment, two sets of the first regions 13A and 14A and the second regions 13B and 14B are provided, but three or more sets may be provided. Although the coil body 2 is provided around the distal end portion 11 of the core shaft 10, it is not limited to the coil body 2, and may be a cylindrical member made of resin or a woven braid.

What is claimed is:

1. A guide wire comprising:
a core shaft having a distal end portion and a proximal end portion,
wherein the distal end portion includes a plurality of sets, each set including, in order from a most distal end side:
a first region having a flat shape in cross section, and
a second region having a circular shape in cross section and having a flexural rigidity higher than a flexural rigidity of the first region, and
flat shape directions of the respective first regions are identical.

2. The guide wire according to claim 1, wherein each of the second regions has a first portion, a second portion located on a proximal end side of the first portion and having an outer diameter larger than an outer diameter of the first portion, and a tapered portion located between the first portion and the second portion and being decreasingly tapered from the second portion toward the first portion.

3. The guide wire according to claim 1, wherein side surfaces of each of the first regions in a plate thickness direction are smooth surfaces, and side surfaces of each of the first regions in a plate width direction are circular arc surfaces.

4. The guide wire according to claim 1, wherein the distal end portion includes a tapered portion located between a most proximal end side of the plurality of sets and the proximal end portion of the core shaft.

5. The guide wire according to claim 1, wherein the distal end portion includes two sets.

6. The guide wire according to claim 1, wherein the distal end portion includes three sets.

7. The guide wire according to claim 1, wherein the flat shape is an oval shape or an elliptical shape having an outline that is entirely composed of curves.

8. The guide wire according to claim 1, wherein the flat shape is an oval shape or an elliptical shape having an outline that does not have a flat surface.
